(19) United States
(12) Patent Application Publication
(10) Pub. No.: US 2017/0035983 A1
(43) Pub. Date: Feb. 9, 2017

(54) APPARATUS FOR DELIVERING AEROSOLIZED MEDICATION

(71) Applicants: Lisa Wilson, Victorville, CA (US); Robert Pantoja, Victorville, CA (US)

(72) Inventors: Lisa Wilson, Victorville, CA (US); Robert Pantoja, Victorville, CA (US)

(21) Appl. No.: 14/818,645

(22) Filed: Aug. 5, 2015

(51) Int. Cl. A61M 16/14; A61M 16/08; A61M 16/04; A61M 16/00; A61M 11/00; A61M 15/00

(52) U.S. Cl. CPC A61M 16/14; A61M 11/007; A61M 15/009; A61M 16/04; A61M 16/0075; A61M 16/0078; A61M 16/085

(57) ABSTRACT

An apparatus for delivering an aerosolized medication to a patient on a ventilation circuit. The apparatus comprise a multi dose metered syringe preloaded with the aerosolized medication such as albuterol. The syringe comprises a Luer lock at a bottom tip, which is removably attachable to a carbon dioxide sampling port of an endotracheal tube. A plunger adapted to be fitted inside the syringe and actuated for delivering a predefined dose of the aerosolized medication into the endotracheal tube, without disconnecting the ventilation circuit. The delivery of aerosolized medication is synchronized with oxygen inspiration for optimal delivery.

### FIELD OF THE DISCLOSURE

The present disclosure generally relates to delivery of aerosolized medication and more specifically relates to an apparatus for delivery of aerosolized medication to intubated patients or patients on mechanical ventilation, without disconnecting or interrupting with the ventilation circuit.

### BACKGROUND

Intrapulmonary drug delivery is one of the preferred ways for administering respiratory medications compared to other routes of administration including systemic administration. Generally medications for intrapulmonary delivery are administered in the form of liquid bolus or aerosol inhalation. Among which, aerosol forms are typically delivered using devices such as metered dose inhalers (MDI), compressed air nebulizers or ultrasonic nebulizers.

Administration of aerosolized medications including bronchodilators such as albuterol, especially to anesthetized or intubated patients poses significant challenges. For example, the effective dose of medication finally reaching the lungs is far lesser than the administered dose. In another example, uneven intrapulmonary distribution of aerosolized medication in patients on mechanical ventilation adds further challenges in providing effective medication.

Therefore there still exists a need for a delivery apparatus for efficiently administering respiratory medication especially aerosolized forms to patients on a ventilation circuit, without interrupting or disconnecting the ventilation circuit.

### SUMMARY OF THE DISCLOSURE

The present disclosure relates to an apparatus for delivering an aerosolized medication to a patient on a ventilation circuit, the apparatus comprising a multi dose metered syringe preloaded with the aerosolized medication, the syringe is attachable to a gas sampling port of an endotracheal tube. A plunger configured to be actuated for delivering a predefined dose of the aerosolized medication into the endotracheal tube, without disconnecting the ventilation circuit. In an embodiment, the syringe is loaded with a pressurized canister comprising aerosolized medication such as a bronchodilator. The plunger is actuated by pressing down to release a predefined dose of medication through a port at bottom tip.

In an embodiment, the syringe comprises a Luer lock at bottom tip, configured to be attached to the carbon dioxide sampling port of the endotracheal tube. The aerosolized medication comprises a bronchodilator such as albuterol, disposed in a pressurized canister or a chamber. The delivery of aerosolized medication is synchronized with oxygen inspiration by the patient, wherein the synchronizing is timed based on ventilation bag or bellow movement.

In another embodiment, the apparatus of the present disclosure is further configured to be removably attached to a mouthpiece for oral inhalation of aerosolized medication, thus permitting the use of the apparatus in an extra-operative setting. The mouthpiece comprises a receptacle for receiving the syringe and an inhalation port covered with a removable cap. In an embodiment, the mouthpiece comprises a metered dose inhaler adapter, wherein the adapter is configured to receive a pressurized canister comprising aerosolized medication. In an embodiment, the aerosolized medication comprises a predefined dose of albuterol.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF SEVERAL VIEWS OF DRAWINGS

FIG. 1 is a schematic view of the apparatus being attached to an endotracheal tube of a patient on mechanical ventilation.

FIG. 2 is a schematic view of the apparatus attached to a mouthpiece for oral inhalation.

### DETAILED DESCRIPTION OF PREFERRED EMBODIMENTS

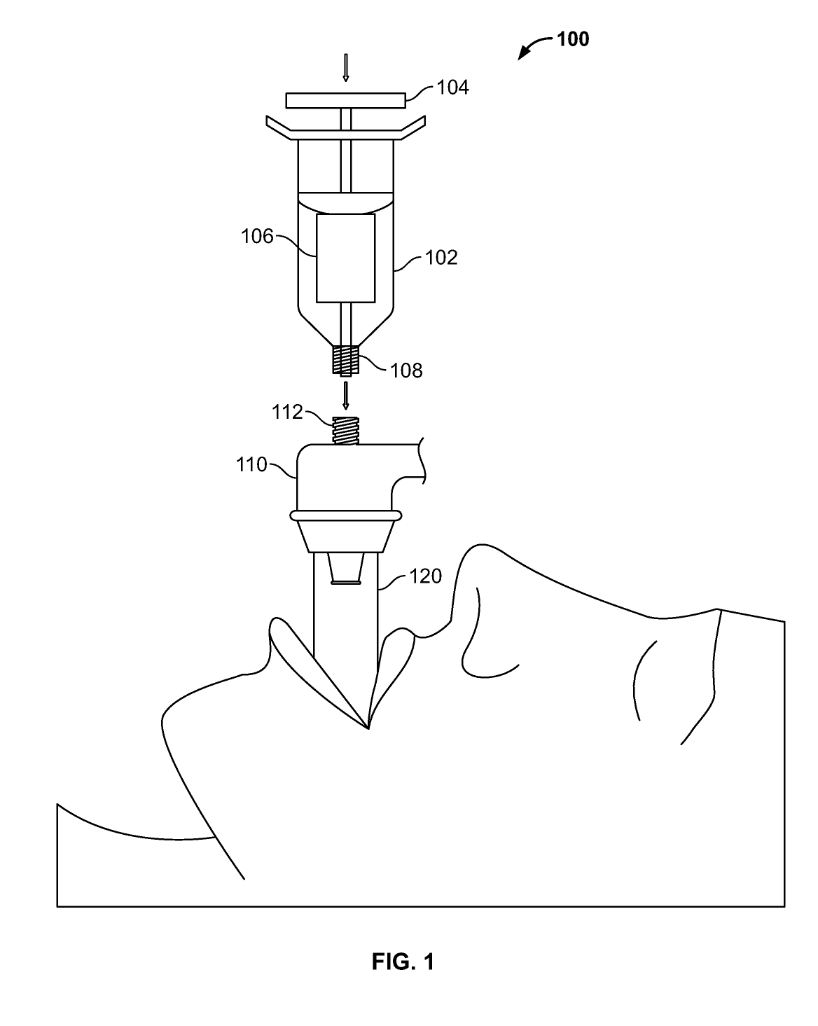

Referring to FIG. 1, which illustrates a schematic view of the apparatus 100 being attached to an endotracheal tube 120 of a patient on mechanical ventilation. The apparatus 100 comprises a syringe 102 preloaded with the aerosolized medication in a chamber or a container 106, the syringe 102 is attachable to an endotracheal tube 120. A plunger 104 configured to be actuated for delivering a predefined dose of the aerosolized medication into the endotracheal tube 120, without disconnecting the ventilation circuit. A connector 110 comprising a gas sampling port 112, is configured to attach the syringe 102 with the endotracheal tube 120 of the patient. The syringe 102 comprises a Luer lock 108 at bottom tip, configured to be fitted to the gas sampling port 112 of the connector 110 which is attached to the endotracheal tube 120.

The syringe 102 may comprise a multi-dose metered syringe for adjusting the dosing of medication to be delivered. The gas sampling port 112 may comprise a carbon dioxide sampling port. In a different embodiment, the bottom tip of the syringe 102 may comprise a Luer taper connection adapted to slip into the carbon dioxide sampling port 112.

The aerosolized medication, for example, albuterol, can be disposed in a pressurized canister or a chamber 106. The delivery of aerosolized medication is synchronized with inspiration of oxygen by the patient using a ventilation bag or bellow, wherein the delivery is synchronized based on ventilation bag or bellow movement for optimum delivery of medication to the patient.

The apparatus 100 of the present disclosure can be attached to a ventilation circuit of an anesthetized patient, for delivering respiratory medication directly into the airway or lungs. The respiratory medication may comprise aerosolized preparations of bronchodilators, anti-inflammatory agents, antibiotics, anti-cholinergics, heparin, steroids and its combinations. In an embodiment, the aerosolized medication may comprise a combination of albuterol and ipratropium (Ex: Combivent®,Duoneb™, etc.,) administered using the apparatus 100 of the present disclosure.

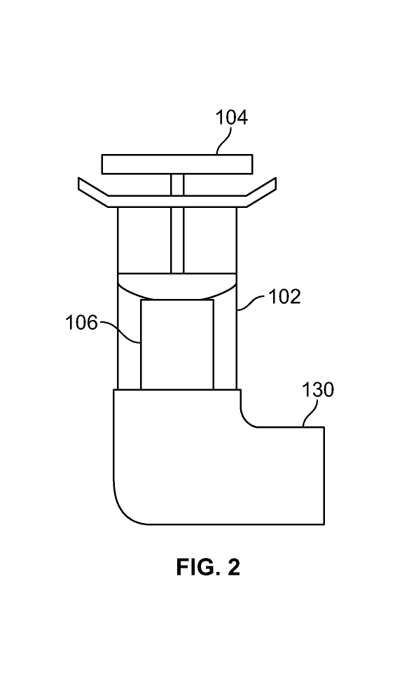

In another embodiment, a mouthpiece 130 is configured to be removably attached to the apparatus 100 as shown in FIG. 2. The mouthpiece 130 may comprise an adapter for oral inhalation of aerosolized medication, thus permitting the use of the apparatus 100 by patients in an extra-operative setting. The mouthpiece 130 comprises a receptacle for receiving the syringe and an inhalation port covered with a removable cap. In an embodiment, the mouthpiece comprises a metered dose inhaler adapter, wherein the adapter is configured to receive a pressurized canister comprising aerosolized medication. For example, the apparatus 100 can be used as an inhaler by pressing the canister downwards and simultaneously inhaling through the mouthpiece 130.

In an exemplary embodiment, the apparatus 100 is compact, hand-held and similar to the size of regular albuterol inhalers. For example, the apparatus 100 may comprise dimensions of 2.5 inches height and 1.5 inches length.

Delivery of aerosolized medication to patients on mechanical ventilation is influenced by a variety of factors including aerosol particle size, airway obstruction, type of delivery device, humidity of inhaled gas, patient-ventilator synchrony etc.

Because of the improved efficiency of delivery of aerosolized medication through metered puffs through the ventilator circuit, the patient require only less metered doses that needs to be given intraoperatively, which in overall prolongs the use of the medication and the metered inhaler overtime.

The apparatus of the present disclosure provides numerous advantages over existing devices, including its simple, compact design and the apparatus can also be placed inside a drug machine slot. The apparatus may comprise multi dose metered syringes preloaded with medication such as albuterol and can be easily attached to existing ventilation circuits through the gas (carbon dioxide) sampling port without any need for additional means like valves, hosing or other attachments such as suction ports which might require to disconnect ventilation temporarily. Whereas the apparatus of the present disclosure allows administering the aerosolized medication without disconnecting or disturbing the ventilation circuit.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents. Some of the figures may be enlarged for demonstration purposes only and are not scalable.

What is claimed is:

1. An apparatus for delivering an aerosolized medication to a patient on a ventilation circuit, the apparatus comprising:
a multi-dose metered syringe preloaded with the aerosolized medication, the syringe is attachable to a gas sampling port of an endotracheal tube; and
a plunger configured to be actuated for delivering a predefined dose of the aerosolized medication into the endotracheal tube, without disconnecting the ventilation circuit.

2. The apparatus of claim 1, wherein the aerosolized medication is disposed in an inhaler or a pressurized canister.

3. The apparatus of claim 1, wherein the aerosolized medication comprises albuterol.

4. The apparatus of claim 1, wherein the syringe further comprises a Luer lock tip configured to be attached to the gas sampling port of the endotracheal tube.

5. The apparatus of claim 1, wherein the delivery of aerosolized medication is synchronized with oxygen inspiration.

6. The apparatus of claim 5, wherein the synchronizing is timed based on ventilation bag or bellow movement.

7. The apparatus of claim 1, wherein the gas sampling port comprises carbon dioxide sampling port.
